(19) United States
(12) Patent Application Publication
(10) Pub. No.: US 2026/0191599 A1
(43) Pub. Date: Jul. 9, 2026

(54) METHOD AND SYSTEM FOR DETERMINING THE POSITION OF A SURGICAL INSTRUMENT

(71) Applicants: Siemens Healthineers AG, Forchheim (DE); Cook Medical Technologies LLC, Bloomington, IN (US)

(72) Inventors: Jonas Frederik Faust, Erlangen (DE); Florian Maier, Buckenhof (DE); Joshua Krieger, Topsfield, MA (US)

(21) Appl. No.: 19/439,355

(22) Filed: Jan. 3, 2026

(30) Foreign Application Priority Data
Jan. 3, 2025 (DE) 10 2025 100 085.9

(51) Int. Cl. A61B 34/20; A61B 90/00

(52) U.S. Cl. CPC A61B 34/20; A61B 90/39; A61B 2034/2051; A61B 2090/3954

(57) ABSTRACT

A method for localizing a two-dimensionally compact metallic object in a patient using a magnetic resonance imaging scanner and a system for performing the method are provided. The metallic object has at least one marker that may be captured by magnetic resonance imaging at a predetermined relative position to the metallic object. In the method, nuclear spins in the vicinity of the compact metallic object are excited, and magnetic resonance data is captured by sampling along a plurality of trajectories. A position of the marker is ascertained from the magnetic resonance data based on captured artifacts and the metallic object. A position of a tip of the metallic object is determined as a function of the position of the at least one marker, and an image of the patient depicting the tip is issued to a user of the magnetic resonance imaging scanner.

This application claims the benefit of German Patent Application No. DE 10 2025 100 085.9, filed on Jan. 3, 2025, which is hereby incorporated by reference in its entirety.

### BACKGROUND

Independent of the grammatical term usage, individuals with male, female, or other gender identities are included within the term.

Magnetic resonance imaging scanners are imaging devices that, for imaging an examination object, align nuclear spins of the examination object with a strong external magnetic field and excite the nuclear spins to precess around this alignment using an alternating magnetic field. The precession or return of the spins from this excited state into a state of lower energy produces an alternating magnetic field in response, which is received via antennas.

Magnetic gradient fields are used to impose spatial encoding on the signals, and this then enables the received signal to be assigned to a volume element. The received signal is then evaluated, and a three-dimensional imaging representation of the examination object is provided. The signal may be received using local receiving antennas (e.g., local coils) that are arranged directly on the examination object in order to achieve a better signal-to-noise ratio.

Magnetic resonance imaging scanners may enable visualization of the body's interior over extended periods without exposing the patient or surgeon to an increased dose of ionizing radiation. However, due to the complex image capturing process and the resulting slow image sequence, magnet resonance imaging is difficult to use for real-time monitoring. In addition, it is necessary to provide real-time visualization of instruments that not only themselves do not produce a magnetic resonance signal, but also inhibit signal capture in the vicinity of the instrument due to their metallic properties.

EP 4465068 A1 discloses a method and a magnetic resonance imaging scanner for localizing metallic objects. In one step of the method, nuclear spins are excited in the vicinity of the compact metallic object by an excitation pulse. Magnetic resonance data is captured by sampling along a plurality of trajectories, where sampling is performed using a bSSFP sequence and the nuclear spins are dephased using a gradient.

A position of a geometric centroid of the compact metallic object is ascertained based on a position of the visual centroid of captured artifacts.

U.S. Pat. No. 11,737,851 B2 discloses a method for providing a marker that may be imaged in a magnetic resonance imaging scanner on a steel catheter wire.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, determination of a position of metallic instruments with a magnetic resonance imaging scanner may be improved.

The method according to the present embodiments is provided for localizing a two-dimensionally compact object in a patient using a magnetic resonance imaging scanner. The two-dimensionally compact object may be an object with two-dimensional dimensions that are significantly smaller than the dimension in the third dimension. Herein, “significantly smaller” provides that the smaller dimensions are smaller by a factor of 5, 20, 50, or more than the dimension in the longer third dimension. In other words, the object is thin and elongated, such as, for example, a biopsy needle or a guide wire for a catheter. Herein, the two-dimensionally compact object is also referred to as a compact object for short.

The compact object has at least one marker that may be acquired with the magnetic resonance imaging scanner during magnetic resonance imaging. For example, the at least one marker may be identified in the magnetic resonance imaging relative to the compact object, even if the latter is metallic (e.g., using a higher intensity and/or higher contrast, at least in one of the sequences used). This may, for example, also relate to improved imaging at specific angles of the compact object to the static magnetic field B0 (e.g., in the case of parallel alignment of a compact metallic object). The marker to be captured is arranged or attached in a predetermined relative position on or to the metallic object.

In one embodiment, the marker is not arranged at the tip of the compact object. Otherwise, for example, in the case of metallic objects, an artifact produced by the discontinuity of the tip, which is free in five spatial directions, superimposes the image of the marker. A marker, which may be arranged at a distance from the tip, is superimposed by an elongated artifact produced by the compact object that may be distinguished from the approximately point-shaped or one-dimensional artifact produced by the marker based on its symmetry. This enables a position of the marker to be determined reliably and accurately in three dimensions.

In one act of the method according to the present embodiments, nuclear spins in the vicinity of the compact object are excited by the magnetic resonance imaging scanner by a radio-frequency pulse to precess around a vector of a magnetic field in the vicinity of the compact object. The magnetic field may be determined by the static magnetic field of a field magnet that may be superimposed by a gradient field for slice selection. The frequency of the radio-frequency pulse results from the magnetic moment of the nuclear spins and the strength of the magnetic field and is referred to as the Larmor frequency. The field strength and duration of the excitation pulse are determined by the degree of excitation or flip angle of the spins to be achieved.

In one embodiment of the method, in one act, in order to suppress the background (e.g., the surrounding tissue), the magnetic resonance imaging scanner applies a magnetic field gradient to dephase nuclear spins located outside the immediate vicinity of the compact metallic object or marker that are therefore not affected by susceptibility changes caused by the metal. A gradient of this type is also referred to as a white marker gradient, since, owing to the Larmor frequency altered by the susceptibility fluctuation, it results in only the immediate vicinity not being dephased by the white marker gradient, or, more precisely, the external white marker gradient counteracts the local susceptibility-induced gradient and reverses the dephasing. Thus, a magnetic resonance signal in the immediate vicinity of the metallic object exhibits high contrast relative to the dephased background.

In a further act, the magnetic resonance imaging scanner captures magnetic resonance data along a plurality of trajectories. Herein, a trajectory refers to a curve or also, for example, to a straight line in k-space along which resonance data is captured or sampled. In one embodiment, sampling takes place at regularly spaced points on the curve. Herein, in the context of the present embodiments, the term “trajectory” does not refer to the sum of all trajectory curves required for imaging, but rather to individual connected sections, also referred to as “readouts.” In radial sampling, the term “trajectory,” for example, refers to individual spokes that pass through the origin in k-space.

In one embodiment, the trajectories are radial or symmetrical to the origin of k-space.

Herein, in one possible embodiment, sampling along the trajectories takes place in the context of a balanced steady-state free precession (bSSFP) sequence, as is known, for example, from https://en.wikipedia.org/wiki/Steady-state_free_precession_imaging.

Herein, the signals from the plurality of samples are used to produce an image of an artifact caused by the compact metallic object and/or the at least one marker in the spatial domain. The magnetic resonance imaging scanner or a separate reconstruction computer may use a reconstruction method to produce an image of the artifacts caused by the compact metallic object from the measured values in the spatial domain.

When using dephasing with the white marker pulse, only artifacts caused by the metallic object or marker are depicted. In one embodiment, dephasing with the white marker gradient eliminates the background of surrounding tissue, which would distort the ascertainment of the instrument position based on the visual centroid of the signal. The artifact caused by the metallic object is thus separated in the resulting image from effects in the vicinity, thus eliminating the need to take such effects into account in subsequent steps.

In a further act, a position of the at least one marker is ascertained from the magnetic resonance data. Due to their special properties (e.g., increased susceptibility), the markers produce a magnetic resonance signal in their vicinity that is also distinct from the signal or artifact produced by the two-dimensionally compact metallic object. In one embodiment, the magnetic resonance imaging scanner or a reconstruction unit separates or segments the artifacts produced by the marker and the two-dimensionally compact object. Herein, the markers may be compact in three dimensions or in an approximately point-like manner so that the artifacts produced by the markers or their centroid also indicate the actual geometric location. Herein, due to the small extension in all dimensions, the marker enters the body completely, and there is no blurring of the position, such as is the case with a two-dimensionally compact object.

With a plurality of markers, in one embodiment, the alignment of the compact object may be defined based on the artifacts produced by at least two markers (e.g., if the compact object is non-metallic). In one embodiment, the artifacts produced by the markers are independent of the alignment of the metallic object relative to the field direction of the static magnetic field, and a signal is produced even when the compact object is aligned parallel to the field.

In a further act of the method, a position of a tip of the compact object is ascertained. This may be done by the magnetic resonance imaging scanner, the reconstruction computer, or a further computing unit or controller. The alignment of the two-dimensionally compact metallic object was already ascertained in the above-described act based on the artifact produced by the compact metallic object and thus defines a straight line in space along which the metallic object is arranged. The ascertained position of the marker and the predetermined relative position of the marker to the metallic object may be used to ascertain its exact position along the alignment or straight line. With knowledge of the geometry of the metallic object, which is also predetermined, the exact position of the tip and other parts of the metallic object are also defined or ascertained in this way.

In the case of a non-metallic compact object, alignment may also be ascertained from the position of at least two markers arranged thereon using a straight line connecting the positions of the two markers. At the same time, the position of the compact object is determined from the position of the markers, which are located at predetermined relative positions on the compact object. Based on the geometry of the compact object, which is also known, the system according to the present embodiments thus also ascertains the position of the tip.

In a further act, the magnetic resonance imaging scanner or a reconstruction unit displays an image of the patient or part of the patient depicting the tip or position of the metallic object to a user on a display.

In one embodiment, the method according to the present embodiments enables a compact object in a patient to be quickly and precisely captured and imaged in terms of both alignment and position.

The system according to the present embodiments has a two-dimensionally compact object and a magnetic resonance imaging scanner with which the object may be captured in a magnetic resonance scan. Herein, the above explanations regarding the magnetic resonance imaging scanner and the metallic object are applicable. Herein, the system may be distributed, for example, by separating controller or a reconstruction unit from the appliances for producing magnetic fields and receiving the magnetic resonance signals (e.g., on a central server or in the cloud).

The system according to the present embodiments is configured to execute the acts of the method according to the present embodiments and in this respect shares its advantages.

In one embodiment of the method, the two-dimensionally compact object has a plurality of markers distributed along a longitudinal extension of the object. Herein, “distributed” may be that the distance between markers is at least sufficient to provide that the markers produce artifacts that may be distinguished from one another when images are captured by the magnetic resonance imaging scanner.

In one embodiment, a first marker is arranged close to the tip in order to produce a signal as soon as possible after insertion into the body. In one embodiment, the second marker is then positioned at a short distance from the first marker so that the second marker may still be distinguished in the magnetic resonance imaging. The distance to the tip and/or to the second marker may, for example, be less than 2 mm, 5 mm, or 10 mm.

In one embodiment, a marker close to the tip enables localization of the marker in a magnetic resonance image as soon as the compact object is inserted into the patient.

In the act of ascertaining a position, positions are ascertained for the plurality of markers as already described for an individual marker.

In the act of ascertaining a position of a tip of the object, this is then determined as a function of or using the position of the plurality of markers. The alignment of the metallic object may be further used in conjunction with the positions of the plurality of markers. The position of the two markers alone already defines an alignment of the object on which the two markers are arranged. This is in advantageous if the compact object itself is non-metallic. The predetermined distance between the first marker and the tip (e.g., the marker is arranged close to the tip) then also defines and ascertains the position of the tip. However, the alignment may also be ascertained solely from the artifact produced by the metallic object, or the redundant directional information from the markers and the metallic object may be used to minimize errors.

If, in one embodiment, more than two markers are arranged on the object, these may be spaced at different distances to their respective neighboring marker or markers, so that they can also be distinguished within the imaging of the magnetic resonance imaging scanner. This enables the individual markers themselves to be distinguished in the imaging. For example, the reconstruction computer or another controller may identify when a marker is not captured in the image. This may be the case if the marker is not yet located in the body or is in an area of the body that does not produce a sufficient magnetic resonance signal (e.g., in an air-filled cavity or on a bone).

In one embodiment, the markers may be arranged at equal distances relative to one another along the two-dimensional compact object.

In one embodiment, the ascertained position of the tip may be checked during the ascertaining by the reconstruction computer or a controller for such signal dropouts and is accordingly discarded, or a warning may be issued to a user. This enables injuries caused by excessively deep insertion to be avoided.

In one embodiment, when ascertaining the position of the tip, based on the relative position, the system identifies and takes into account which marker is not providing a signal and then ascertains and displays the correct position of the tip based on the position of the ascertainable markers.

The explanations apply equally to the embodiments of the system with which the method is performed.

The above-described properties, features, and advantages of this invention and the manner in which these are achieved will become clearer and more plainly comprehensible in conjunction with the following description of the example embodiments explained in more detail in conjunction with the drawings.

### BRIEF DESCRIPTION OF THE DRAWINGS

FIG. 1 is a schematic representation of an embodiment of a system for executing a method according to the present embodiments;

FIG. 2 is a schematic representation of an example of an object of a system according to the present embodiments;

FIG. 3 is a schematic flowchart of an embodiment of the method.

### DETAILED DESCRIPTION

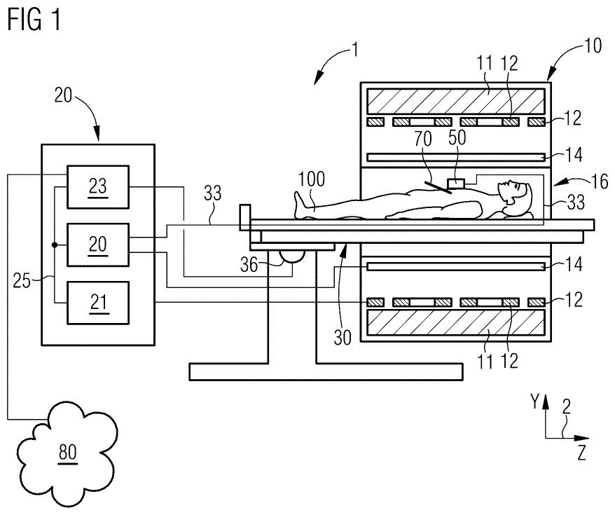

FIG. 1 is a schematic representation of an embodiment of a system 200 for executing a method according to the present embodiments.

The system 200 includes a magnet unit 10 that has a field magnet 11 that produces a static magnetic field B0 for aligning nuclear spins of test specimens or a patient 100 in a receiving region. The receiving region is characterized by an extremely homogeneous static magnetic field B0, where the homogeneity, for example, relates to the magnetic field strength or the magnitude. The receiving region is almost spherical in shape and arranged in a patient tunnel 16 that extends in a longitudinal direction 2 through the magnet unit 10.

A patient couch 30 may be moved in the patient tunnel 16 by a positioning unit 36.

The field magnet 11 may be a superconducting magnet that may provide magnetic fields with a magnetic flux density of up to 3T, or even more in the latest devices. However, permanent magnets or electromagnets with normally conducting coils may be used for lower field strengths.

The magnet unit 10 further includes gradient coils 12 that, for spatial differentiation of the imaging regions captured in the examination volume, are configured to superimpose variable magnetic fields onto the magnetic field B0 in three spatial directions. The gradient coils 12 may be coils made of normally conducting wires that may produce mutually orthogonal fields in the examination volume.

The magnet unit 10 also has a body coil 14 configured to radiate a radio-frequency signal supplied via a signal line into the examination volume and to receive resonance signals emitted by the patient 100 and output the resonance signals via a signal line. Hereinafter, the term “transmitting antenna” refers to an antenna via which the radio-frequency signal for exciting the nuclear spins is emitted. This may be the body coil 14, but also a local coil 50 with a transmitting function.

A control unit 20 supplies the magnet unit 10 with the various signals for the gradient coils 12 and the body coil 14 and evaluates the received signals.

Thus, the control unit 20 has a gradient actuator 21 configured to supply the gradient coils 12 with variable currents via supply lines that provide the desired gradient fields in the examination volume in a temporally coordinated manner.

The control unit 20 also has a radio-frequency unit 22 configured to produce a radio-frequency pulse with a predetermined temporal profile, amplitude, and spectral power distribution for exciting a magnetic resonance of the nuclear spins in the patient 100. Herein, pulse powers in the kilowatt range may be achieved. The excitation signals may be radiated into the patient 100 via the body coil 14 or also via a local transmitting antenna.

A controller 23 communicates with the gradient controller 21 and the radio-frequency unit 22 via a signal bus 25.

A local coil 50 is arranged on the patient 100 and connected to the radio-frequency unit 22 and its receiver via a connecting line 33. However, in one embodiment, the body coil 14 may be a receiving antenna within the present embodiments.

A compact object 70 (e.g., a biopsy needle or a catheter or catheter guide wire) may be inserted into the patient 100. The compact object 70 is two-dimensionally compact (e.g., has a longitudinal extension, and the dimensions transverse to the longitudinal extension are significantly smaller than the dimension in the longitudinal extension, such as by a factor greater than 5 or 10). The dimensions transverse to the longitudinal extension are so small that the width of the resulting artifact is substantially independent of these dimensions. For example, the dimensions transverse to the longitudinal extension are less than 5 mm, 2 mm, or 1 mm.

The object 70 is metallic and produces artifacts during magnetic resonance imaging, since alternating electromagnetic fields are attenuated. Due to its susceptibility, which differs from that in the vicinity, the metal also brings about a change in the static and quasi-static magnetic fields (e.g., the magnetic field B0 and the gradient fields). This results in same magnetic field values occurring at different geometric locations in the vicinity of the object, which are imaged to a common point via the spatial encoding. At the same time, in conventional image capturing, the vicinity of the object is distorted by the variation in the magnetic field and transposed into a different position relative to the surrounding organs.

The compact object 70 has at least one marker 75. During image capture with the magnetic resonance imaging scanner 1, the at least one marker 75 produces a magnetic resonance signal or artifact that may be distinguished from the metallic object 70 in the sequence used (e.g., in its shape and/or intensity). In one embodiment, the artifact is point-like in the sense that the artifact allows localization of the marker 75 by having a low spatial extent (e.g., it is limited to a radius of smaller than 10 mm, 5 mm, or 2 mm around the location of the marker in the resulting image).

The magnetic resonance imaging scanner 1 may also be connected via a signal link to an external signal processing resource, such as a cloud 80, as part of the system in which parts of the method according to the present embodiments are executed (e.g., image reconstruction and the ascertainment of the positions and alignment described below).

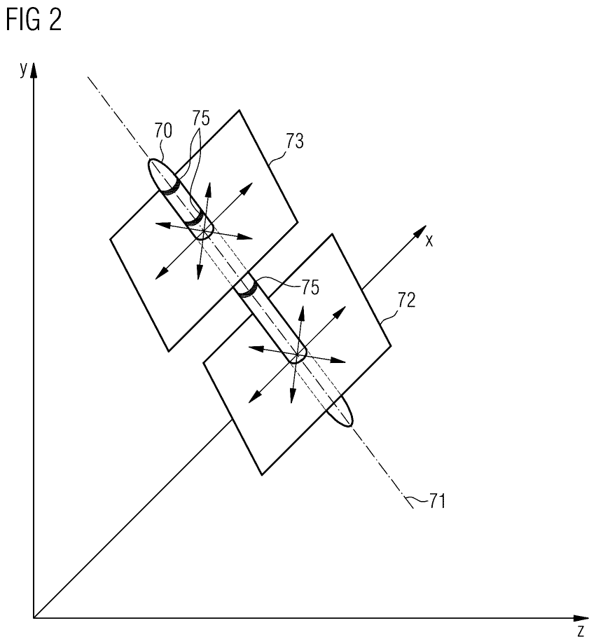

FIG. 2 depicts an example of a compact object 70 of the system 200 of the present embodiments. The object 70 in FIG. 2 extends along an axis 71, where the extension along the axis is significantly greater than the dimensions of the object 70 transverse to the axis 71. In the context of the present embodiments, an object 70 of this kind is referred to as linear and compact in two dimensions. This is, for example, evident from the sequence of the method shown in FIG. 4, which largely corresponds to that for the compact object 70 in three dimensions.

The orientation of a linear object 70 of this kind may be determined precisely if the axis 71, which is a straight line, is determined by two points. This may be achieved by ascertaining the points of intersection of the linear object 70 with two planes that are spaced apart from one another.

For this purpose, the magnetic resonance imaging scanner 1 is to determine these two planes in one step. The magnetic resonance imaging scanner 1 may, for example, have stored data from a treatment plan or an operator input that indicates a rough aberrant location and position of the object 70. In one embodiment, with an aberrant conventional sequence, the magnetic resonance imaging scanner 1 determines the location roughly. Based on this location, the magnetic resonance imaging scanner 1 may use linear algebra to ascertain a first plane 72 and a second plane 73 that in each case have a definite point of intersection with the object 70 (e.g., by taking account of safety distances between the planes and the extreme coordinates of the object 70). The planes may be parallel to one another and perpendicular to the axis 71, but other arrangements may also be provided as long as the planes do not coincide or are aligned parallel to the axis 71.

Three markers 75 are arranged on the object 70. The markers 75 are in each case spaced at different distances from one another. In one embodiment, at least one marker 75 is arranged close to a proximal tip or end of the metallic object 70.

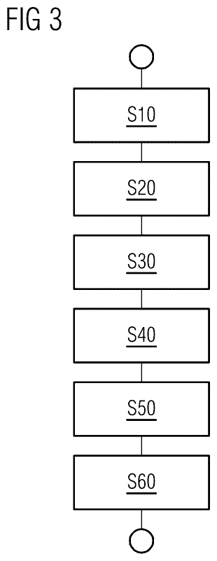

FIG. 3 shows a schematic flowchart of an embodiment of a method.

In one act S10, nuclear spins in the vicinity of the compact object 70 are excited by an excitation pulse.

In one embodiment, excitation is performed using a bSSFP sequence. The bSSFP sequence is characterized by the fact that the zero-th gradient moment is reversed on all axes (e.g., by playing out all gradients that are played out after the excitation pulse before the next excitation with opposite polarity).

In a further act S20, the magnetic resonance imaging scanner 1 captures magnetic resonance data. In an embodiment, sampling takes place along a radial trajectory in k-space or along a trajectory that extends symmetrically through the origin of k-space.

Herein, in one embodiment, the nuclear spins are dephased in act S21 using a gradient in order to mask out the background of the body. Only the immediate vicinity of the object 70 remains unaffected by the magnetic field changes caused by the susceptibility jump, since the duration and/or strength of the gradient is selected precisely such that the gradient dephases the magnetic resonance signals in the unaffected, more distant tissue. The magnetic resonance imaging scanner then captures MR signals with a sampling scheme along a radial trajectory in k-space.

In one embodiment, the magnetic resonance imaging scanner 1 also captures MR signals with a sampling scheme along the radial trajectory 90 in the opposite direction in k-space.

In act S30, a position of the marker or markers 75 is ascertained from the received magnetic resonance signals ascertained. For example, a centroid may be determined from the brightness values in the image space. In one embodiment, the artifacts produced by markers 75 and the metallic object 70 are separated or segmented beforehand. The separation or even the entire ascertaining of the position may be performed by a trained neural network.

In a further act S40, the alignment of the compact object 70 is ascertained based on captured artifacts produced by the compact object 70 and/or the at least one marker in an image reconstructed from the magnetic resonance data.

If there is only one marker, in one embodiment, a straight line may be fitted to the elongated artifacts produced by the compact metallic object 70 in order to ascertain the orientation of the metallic object 70. The ascertained alignment may then be checked against the position of the at least one marker 75 or minimized within the framework of an optimization method.

If there are at least two markers 75, the positions of the markers 75 may also be used to ascertain the alignment of the compact object 70 by a straight line passing through the two markers 75. In this case, it is not necessary for the compact object 70 to be metallic. A combination of both methods in which the position of the at least two markers 75 or the straight line through the markers 75 is compared with the ascertained alignment of the compact metallic object 70 or the deviation of both types of data from the alignment is minimized using an optimization method may be provided. In one embodiment, a neural network may be trained to ascertain the alignment and/or positions of the compact object 70 and of the marker or markers 75 from the image data or directly from k-space-data.

In act S50, a position of a tip of the compact object is ascertained based on the alignment of the compact object 70 and the position of the at least one marker 75. This is done using information about the predetermined relative position of the marker 75 on the compact object 70. Since the absolute position of the marker 75 and the alignment of the metallic object 70 is known from the previous steps, as well as the geometry of the metallic object 70, the system 200 or a controller of the system 200 may ascertain the absolute position of the tip of the compact object 70 and, in act S60, issue the absolute position of the tip of the compact object 70 in an image of the patient depicting the tip to a user of the magnetic resonance imaging scanner 1.

In one embodiment of the system 200 according to the present embodiments, the compact object 70 has at least three markers 75 arranged at different distances to one another so that the markers 75 may be identified in an image based on the distances to one another. In this way (e.g., in cases where not all markers 75 produce a signal because they are still outside the body or are in a vicinity without signal generation, such as a gas-filled cavity), the position of the compact object 70 may be ascertained reliably. Based on the different distances, the controller may ascertain which markers are imaged and thus ascertain the absolution position of the compact object 70 and, for example, the tip using the known relative positions of the markers 75.

Although the invention has been illustrated and described in detail by the example embodiments, the invention is not restricted by the disclosed examples, and other variations may be derived herefrom without departing from the scope of protection of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for localizing an object that is two-dimensionally compact in a patient using a magnetic resonance imaging scanner, wherein the object has at least one marker that is capturable by magnetic resonance imaging at a predetermined relative position to the object, the method comprising:
exciting nuclear spins in the vicinity of the object using an excitation pulse;
capturing magnetic resonance data, the capturing of the magnetic resonance data comprising sampling along a plurality of trajectories;
ascertaining a position of the at least one marker from the magnetic resonance data;
ascertaining a position of a tip of the object taking into account the position of the at least one marker; and
issuing an image of the patient depicting the tip to a user of the magnetic resonance imaging scanner.

2. The method of claim 1, wherein the at least one marker comprises at least two markers, and the position of the tip of the compact object is ascertained as a function of the positions of the at least two markers.

3. The method of claim 1, wherein the object is metallic,
wherein the method further comprises ascertaining an alignment of the metallic object based on captured artifacts produced by the metallic object, the at least one marker in an image reconstructed from the magnetic resonance data, or a combination thereof, and
wherein ascertaining the position of the tip of the metallic object comprises ascertaining the position of the tip of the metallic object as a function of the alignment of the metallic object.

4. The method of claim 3, wherein the sampling is performed using a white marker pulse, a bSSFP sequence, or the white marker pulse and the bSSFP sequence.

5. The method of claim 1, wherein the plurality of trajectories are radial, symmetrical, or radial and symmetrical to the origin of k-space.

6. The method of claim 1, wherein the at least one marker comprises a plurality of markers distributed along a longitudinal extension of the object,
wherein ascertaining the position of the at least one marker comprises ascertaining positions for the plurality of markers, and
wherein ascertaining the position of the tip of the object comprises ascertaining the position of the tip of the object based on the position of the plurality of markers or an alignment in conjunction with the position of the plurality of markers.

7. The method of claim 6, wherein the at least one marker comprises at least three markers spaced at different distances to their respective neighboring markers, and
wherein ascertaining the position of the tip of the object comprises ascertaining or checking the position of the tip of the objection as a function of the relative positions of the markers to one another.

8. A system comprising:
a magnetic resonance imaging scanner; and
a metallic object that is two-dimensionally compact, wherein the metallic object comprises at least one marker that is capturable by magnetic resonance imaging at a predetermined relative position to the metallic object,
wherein the system is configured to:
excite nuclear spins in the vicinity of the metallic object using an excitation pulse from a radio-frequency unit;
capture magnetic resonance data with the magnetic resonance imaging scanner with sampling along a plurality of trajectories;
ascertain a position of the at least one marker based on artifacts produced by the at least one marker in an image reconstructed from the magnetic resonance data;
ascertain a position of a tip of the metallic object as a function of the position of the at least one marker; and
issue an image of the patient depicting the tip to a user of the magnetic resonance imaging scanner on an output device.

9. The system of claim 8, wherein the system is further configured to:
ascertain an alignment of the metallic object based on artifacts produced by the metallic object in the image reconstructed from the magnetic resonance data; and
determine the position of the tip of the metallic object as a function of the alignment of the metallic object.

10. The system of claim 8, wherein the metallic object has a plurality of markers distributed along a longitudinal extension of the metallic object,
wherein the system is configured to ascertain positions for the plurality of markers, and
wherein the system is configured to ascertain a position of the tip of the metallic object based on the positions of the plurality of markers.

11. The system of claim 8, wherein the at least one marker comprises at least three markers spaced at different distances to their respective neighboring markers, and
wherein the system is configured to ascertain or check the position of the tip of the metallic object as a function of the relative positions of the markers.

12. The system as claimed of claim 8, wherein the system is configured to perform the sampling using a white marker pulse and a bSSFP sequence.

13. The system of claim 8, wherein the trajectories are radial, symmetrical, or radial and symmetrical to the origin of k-space.

14. In a non-transitory computer-readable storage medium that stores instructions executable by one or more processors to localize an object that is two-dimensionally compact in a patient using a magnetic resonance imaging scanner, wherein the object has at least one marker that is capturable by magnetic resonance imaging at a predetermined relative position to the object, the instructions comprising:
exciting nuclear spins in the vicinity of the object using an excitation pulse;
capturing magnetic resonance data, the capturing of the magnetic resonance data comprising sampling along a plurality of trajectories;
ascertaining a position of the at least one marker from the magnetic resonance data;
ascertaining a position of a tip of the object taking into account the position of the at least one marker; and
issuing an image of the patient depicting the tip to a user of the magnetic resonance imaging scanner.

15. The non-transitory computer-readable storage medium of claim 14, wherein the at least one marker comprises at least two markers, and the position of the tip of the compact object is ascertained as a function of the positions of the at least two markers.

16. The non-transitory computer-readable storage medium of claim 14, wherein the object is metallic,
wherein the method further comprises ascertaining an alignment of the metallic object based on captured artifacts produced by the metallic object, the at least one marker in an image reconstructed from the magnetic resonance data, or a combination thereof, and
wherein ascertaining the position of the tip of the metallic object comprises ascertaining the position of the tip of the metallic object as a function of the alignment of the metallic object.

17. The non-transitory computer-readable storage medium of claim 16, wherein the sampling is performed using a white marker pulse, a bSSFP sequence, or the white marker pulse and the bSSFP sequence.

18. The non-transitory computer-readable storage medium of claim 14, wherein the plurality of trajectories are radial, symmetrical, or radial and symmetrical to the origin of k-space.
